(19) United States
(12) Patent Application Publication
(10) Pub. No.: US 2016/0193381 A1
(43) Pub. Date: Jul. 7, 2016

(54) HEMOSTATIC PRODUCTS

(71) Applicant: ST. TERESA MEDICAL, INC., WOODBURY, MN (US)

(72) Inventors: Curtis Olson, St. Paul, MN (US); Timothy Floyd, Eagle, ID (US); Philip Messina, Woodbury, MN (US)

(21) Appl. No.: 14/988,522

(22) Filed: Jan. 5, 2016

Related U.S. Application Data
(60) Provisional application No. 62/100,268, filed on Jan. 6, 2015.

(51) Int. Cl. A61L 24/00; A61L 24/04

(52) U.S. Cl. CPC A61L 24/0015; A61L 24/0036; A61L 24/0042; A61L 24/043; A61L 2300/254; A61L 2300/252; A61L 2300/418; A61L 2400/04; A61L 2300/802; A61L 2300/604

(57) ABSTRACT

A method of forming a hemostatic product. A porous base is prepared that is capable of substantially dissolving when applied to a bleeding wound. A hemostatic agent is mixed with at least one salt to prepare a hemostatic mixture. The hemostatic mixture is dispersed on the porous base to form the hemostatic product.

### REFERENCE TO RELATED APPLICATION

This application claims priority to U.S. Provisional Application Number 62/100,268, which was filed on Jan. 6, 2015, the contents of which are incorporated herein by reference.

### FIELD OF THE INVENTION

The invention relates generally to products having hemostatic characteristics. More particularly, the invention relates to stabilizers for use in hemostatic products.

### BACKGROUND OF THE INVENTION

The body's natural response to stem bleeding from a wound is to initiate blood clotting via a complex process known as the coagulation cascade. The cascade involves two pathways that ultimately lead to the production of the enzyme thrombin, which catalyzes the conversion of fibrinogen to fibrin.

Fibrin is then cross-linked to form a clot, resulting in hemostasis. For wounds that are not severe, and in individuals that have no countervening conditions, the body is usually able to carry out this process efficiently in a manner that prevents excessive loss of blood from the wound. However, in the case of severe wounds, or in individuals in whom the clotting mechanism is compromised, this may not be the case.

For such individuals, it is possible to administer components of the coagulation cascade, especially thrombin and fibrinogen, directly to the wound to bring about hemostasis. Bandaging of bleeding wounds is also a usual practice, in part to isolate and protect the wounded area, and also to provide a means to exert pressure on the wound, which can also assist in controlling bleeding.

While these methods may be carried out satisfactorily in cases of mild trauma or under conditions of “controlled” wounding (e.g. surgery), many situations in which such treatments are most needed are also those in which it is the most difficult to provide them. Examples of such wounds include, for example, those inflicted during combat or unanticipated wounds that occur as the result of accidents. In such circumstances, survival of the wounded individual may depend on stopping blood loss from the wound and achieving hemostasis during the first few minutes after injury. Unfortunately, given the circumstances of such injuries, appropriate medical intervention may not be immediately available.

In particular, the treatment of penetrating wounds such as bullet wounds or some wounds from shrapnel is problematic. This is due to the difficulty in placing a hemostatic product and/or therapeutic agents at the actual site of injury, which includes an area that is well below the body surface and difficult or impossible to access using conventional techniques.

Jiang et al. in Biomacromolecules, v. 5, p. 326-333 (2004) teaches electrospun dextran fibers. Agents associated with the fibers (e.g. BSA, lysozyme) are directly electrospun into the fibers. The fibers may also include other polymers electrospun with the dextran.

Jiang et al. in Journal of Biomedical Materials Research Part B: Applied Biomaterials, p. 50-57 (2006) discloses electrospun fibers that are a composite of poly(c-caprolactone) as a shell and dextran as a core. These fibers provide the slow release of agents (bovine serum albumin, BSA) that are also electrospun into the fibers.

Smith et al., U.S. Pat. No. 6,753,454, discloses electrospun fibers comprising a substantially homogeneous mixture of a hydrophilic polymer and a polymer that is at least weakly hydrophobic, which may be used to form a bandage. The bandage may comprise active agents (e.g. dextran). However, the disclosed fibers are not readily soluble in liquid.

MacPhee et al., U.S. Pat. No. 6,762,336, teaches a hemostatic multilayer bandage that comprises a thrombin layer between two fibrinogen layers. The bandage may contain other resorbable materials such as glycolic acid or lactic acid based polymers or copolymers. Neither electrospun fibers nor dextran fibers are taught as components of the bandage.

Smith et al., U.S. Pat. No. 6,821,479, teaches a method of preserving a biological material in a dry protective matrix, the matrix comprising fibers such as electrospun fibers. One component of the fibers may be dextran, but homogeneous dextran fibers are not described.

Cochrum et al., U.S. Pat. No. 7,101,862, teaches hemostatic compositions and methods for controlling bleeding. The compositions comprise a cellulose-containing article (e.g. gauze) to which a polysaccharide is covalently or ionically crosslinked. The crosslinked polysaccharide may be dextran. However, the compositions are not electrospun and exogenous clotting agents are not included in the compositions.

Wnek et al., U.S. Patent Publication No. 2004/0018226, discloses fibers produced by an electroprocessing technique such as electrospirming. The fibers comprise enclosures within the fibers for containing substances that are not miscible with the fibers. Dextran is not taught as a fiber component.

Fisher et al., U.S. Patent Publication No. 2007/0160653, teaches a hemostatic textile comprising hemostatic factors (e.g. thrombin, fibrinogen) but the fibers are formed from electrospun glass plus a secondary fiber (e.g. silk, ceramic, bamboo, jute, rayon, etc.).

Carpenter et al., U.S. Patent Publication No. 2008/0020015, teaches wound dressing comprised of various biodegradable polymers and hydrogels having allogenic or autologous precursor cells (e.g. stem cells) dispersed within the polymers. The polymers may be prepared by electrospinning, and one polymer component may be dextran. However, the polymers cannot be immediately soluble upon contact with liquid, as they must provide a scaffolding for delivery of the cells over time, even though the polymers eventually biodegrade in situ.

Li et al., U.S. Patent Publication No. 2008/0265469, describes electrospun nanofibers that may include dextran. However, the nanofibers are not described as readily soluble in liquids.

Eskridge et al., U.S. Patent Publication No. 2009/0053288, teaches a woven hemostatic fabric comprised of about 65% fiberglass yarn and about 35% bamboo yarn. The fiberglass component may be electrospun, and hemostatic factors such as thrombin may be associated with the fabric, e.g. by soaking the material in a solution of thrombin. This document indicates that dextran may be added as a hygroscopic agent.

There is an ongoing need to provide improved methods and means to initiate blood clotting in wounds to stop or at least slow blood loss. In particular, there is an ongoing need to improve the capability to readily promote hemostasis in severe wounds in a facile manner, especially under circumstances where immediate treatment by medical personnel is limited or unavailable.

Bowlin et al., U.S. Patent Publication No. 2011/0150973, discloses a method of delivering one or more agents of interest to a location of interest. The method includes applying or delivering to a location of interest a hemostatic product. The hemostatic product includes electrospun dextran fibers that dissolve upon contact with liquid. The hemostatic product also includes one or more agents of interest associated with said electrospun dextran fibers. Applying or delivering results in dissolution of the electrospun dextran fibers in liquid at the location of interest to thereby release the one or more agents of interest into the liquid.

### SUMMARY OF THE INVENTION

An embodiment of the invention is directed to a method of forming a hemostatic product. A porous base is prepared that is capable of substantially dissolving when applied to a bleeding wound. A hemostatic agent is mixed with at least one salt to prepare a hemostatic mixture. The hemostatic mixture is dispersed on the porous base to form the hemostatic product.

Another embodiment of the invention is directed to a method of forming a hemostatic product. A porous base is prepared that is capable of substantially dissolving when applied to a bleeding wound. A hemostatic agent is mixed with at least one biologically tolerable polymer to prepare a hemostatic mixture. The hemostatic mixture is dispersed on the porous base to form the hemostatic product.

Another embodiment of the invention is directed to a method of forming a hemostatic product. A porous base is prepared that is capable of substantially dissolving when applied to a bleeding wound. A thrombin component is provided. A fibrinogen component is provided. A particle size of at least one of the thrombin component and the fibrinogen component is reduced to less than about 0.75 millimeters. The thrombin component and the fibrinogen component are mixed to form a hemostatic mixture. The hemostatic mixture is dispersed on the porous base to form the hemostatic product.

### DETAILED DESCRIPTION OF THE PREFERRED EMBODIMENTS

An embodiment of the invention is directed to a system for providing hemostasis in a person or animal. When the hemostatic product is applied to the injury site, the materials used to fabricate the hemostatic product dissolve to thereby release the materials to the injury site and provide the hemostatic effect.

In some embodiments of the invention, only electrospun dextran fibers are utilized and thus after clot formation, there is no need to disturb the clot to remove hemostatic product components, since none remain at the site. The hemostatic product thereby does not leave any residual foreign bodies that elicit foreign body reactions or act as a nidus for infection. Furthermore, the hemostatic product does not contain any xenoproteins, which have the potential of eliciting immune reactions in persons on which the hemostatic product is used.

In other embodiments, as described below, the hemostatic product may include other materials such as support or backing material, which, after initial rapid application of the hemostatic product, may later be removed for further treatment of the wound by conventional methods.

The system generally includes a hemostatic product having a base to which at least one hemostatic agent is associated. In certain embodiments, the base is fabricated from electrospun dextran and the hemostatic agent is thrombin and/or fibrinogen.

Electrospinning is a non-mechanical processing strategy and can be scaled to accommodate the large volumes necessary to meet the needs of commercial processing.

Additional details on the electrospinning process are provided in U.S. application Ser. No. 12/937,322, the contents of which are incorporated herein by reference.

In certain embodiments, the base used in the hemostatic products is formed of substantially homogeneous spun dextran. The amount of dextran used in each hemostatic product can vary depending on the size of hemostatic product that is being manufactured, with typical hemostatic product formulations using from about 5-10 grams of dextran (usually 100,000-200,000 Mr) per hemostatic product.

Of more consequence is the concentration of dextran in the solution from which the fibers are electrospun. Generally, a solution of dextran for electrospinning will be of a concentration in the range of between about 0.1 and about 10 grams per milliliters of solvent. In other embodiments, the dextran concentration is between about 0.5 and about 5 grams per milliliter, and usually such a solution is at a concentration of about 1 gram per milliliter, which is about 0.15 milligrams. A preferred range would be from about 0.9 to about 1.1 grams of dextran per milliliter of solution that is to be spun.

Those of skill in the art will recognize that a variety of liquid solvents exist in which it is possible to dissolve dextran. However, superior results for electrospinning dextran are generally achieved when the solvent is water, especially deionized or distilled or deionized, distilled (ddH2O) or other forms of relatively pure water. In addition, there are no negative interactions during use of the hemostatic product associated with water remaining in the hemostatic product and there is far less environmental impact associated with the use of water as compared to many other solvents.

The area (length and width) of the hemostatic product of the invention can vary widely and can be adjusted by adjusting spinning parameters. In addition, the mats of dextran fibers can be cut to a desired size after spinning. Generally, the hemostatic product will be from about 0.5 centimeters or less to about 30 centimeters or more in length and/or width, but larger or smaller sizes are also contemplated.

The thrombin and/or fibrinogen that are associated with the hemostatic product are in forms that are biologically active when they come into contact with blood. Hence, upon dissolution, the thrombin acts on the fibrinogen, converting it to fibrin, which then forms a clot within the wound to thereby staunch the flow of blood.

In certain embodiments, the thrombin and fibrinogen may be derived from human sources. In other embodiments, the thrombin and fibrinogen are salmon thrombin and fibrinogen. Advantages of using salmon as a source of these materials include but are not limited to the lack of concern about transmission of etiologic agents (e.g. viruses) that may occur when human and other mammalian sources of thrombin or fibrinogen (e.g. bovine) are used.

The components used in fabricating the hemostatic product should be selected to be the same as components found in a living body where the hemostatic product is to be used. Alternatively, the components used in fabricating the hemostatic product are compatible with and readily broken down when the hemostatic product is used on or in a living body. Using such a process minimizes complications associated with components of the hemostatic product not being promptly being broken down as such a process could cause inflammation in the living body. The only thing that remains after the use of the hemostatic product is the clot, which most living bodies are adapted to degrade over time.

A fibrinogen mixture was prepared by mixing fibrinogen with at least one salt. Mixing the fibrinogen with the at least one salt enhances the stability of the fibrinogen during storage prior to fabricating the hemostatic product as well as after the hemostatic product is fabricated.

In certain embodiments, the fibrinogen mixture comprises fibrinogen that is derived from a human source. The fibrinogen may be provided in the fibrinogen mixture at a concentration of between about 40 percent by weight and about 70 percent by weight. In other embodiments, the fibrinogen is provided in the fibrinogen mixture at a concentration of between about percent by weight 50 percent by weight and about 60 percent by weight.

In certain, embodiments, the at least one salt is sodium chloride. The at least one salt is provided in the fibrinogen mixture at a concentration of between about 15 percent by weight and about 50 percent by weight. In other embodiments, the at least one salt is provided in the fibrinogen mixture at a concentration of between about 25 percent by weight and about 35 percent by weight.

The at least one salt may also include trisodium citrate that is provided in the fibrinogen mixture at a concentration of between about 10 percent by weight and about 30 percent by weight. In other embodiments, the trisodium citrate is provided in the fibrinogen mixture at a concentration of between about 15 percent by weight and about 25 percent by weight.

A thrombin mixture was prepared by mixing thrombin with at least one salt and at least one buffer. Mixing the thrombin with the at least one salt and the at least one buffer enhances the stability of the thrombin prior to fabricating the hemostatic product as well as after the hemostatic product is fabricated.

In certain embodiments, the thrombin comprises thrombin that is derived from a human source. The thrombin may be provided in the thrombin mixture at a concentration of between about 0.5 percent by weight and about 2.5 percent by weight. In other embodiments, the thrombin is provided in the thrombin mixture at a concentration of between about 1.0 percent by weight and about 1.5 percent by weight.

In certain embodiments, the at least one salt is sodium chloride. The at least one salt is provided in the thrombin mixture at a concentration of between about 50 percent by weight and about 70 percent by weight. In other embodiments, the at least one salt is provided in the thrombin mixture at a concentration of between about 60 percent by weight and about 65 percent by weight.

In certain embodiments, the at least one buffer is bis(2-hydroxyethyl)amino-tris(hydroxymethyl)methane. The at least one buffer is provided in the thrombin mixture at a concentration of between about 15 percent by weight and about 35 percent by weight. In other embodiments, the at least one buffer is provided in the thrombin mixture at a concentration of between about 25 percent by weight and about 35 percent by weight.

The thrombin mixture may also include a biologically tolerable polymer. An example of one such biologically tolerable polymer is poly(ethylene glycol). The poly(ethylene glycol) may have a molecular weight of between about 5,000 and about 20,000. In certain embodiments, the poly(ethylene glycol) has a molecular weight of between about 7,000 and about 10,000.

The biologically tolerable polymer is provided in the thrombin mixture at a concentration of between about 5 percent by weight and about 25 percent by weight. In other embodiments, the biologically tolerable polymer is provided in the thrombin mixture at a concentration of between about 8 percent by weight and about 20 percent by weight. In still other embodiments, the concentration of the biologically tolerable polymer in the thrombin mixture is between about 13 percent by weight and about 20 percent by weight.

The components used to fabricate the thrombin mixture are mixed together until a substantially homogeneous mixture is prepared. The mixing process should be sufficiently gentle so that undue stress is not placed on the thrombin particles as such stress could impact the efficacy of the thrombin during the use of the hemostatic product.

One of the aspects of obtaining enhanced efficacy of the hemostatic product is preparing a substantially homogeneous hemostatic mixture prior to applying the hemostatic mixture to the support material such as electrospun dextran while at the same time minimizing damage to the proteins used in the hemostatic mixture.

An important aspect of preparing the substantially homogeneous hemostatic mixture is ensuring that the proteins used in fabricating the hemostatic mixture do not form into large clumps because such clumps generate uneven concentrations of the proteins. One technique that has been found to be effective in preventing the formation of large clumps while minimizing damage to the proteins is by passing the components that are used in fabricating the hemostatic mixture through a sifter. In certain embodiments, the sifter is an automatic sifter having a mesh size of between about 0.2 millimeters and about 2.0 millimeters. In other embodiments, the sifter has a mesh size of between about 0.25 millimeters and about 0.75 millimeters. Such automatic sifters may incorporate a cylindrical screen and the materials are urged against the screen using centrifugal motion.

Thereafter, the shifted materials are placed into a mixer. The mixer, the mixing conditions and the mixing duration should be selected to substantially homogeneously mix the components in the hemostatic mixture. In certain embodiments, the mixer utilizes multiaxial movement such as rotating and pivoting of the vessel in which the components are placed. The rate at which the mixer moves is selected to be sufficiently fast to cause mixing of the components while minimizing damage to the proteins during the mixing process.

The mixing can be performed for an extended period of time to ensure that the hemostatic mixture is substantially homogeneous. The mixing may be done for more than 60 minutes. In certain embodiments, the mixing is done for between about 120 and about 240 minutes.

Once the mixing of the hemostatic mixture has been completed, the hemostatic mixture may be packaged prior to using the hemostatic mixture in fabricating the hemostatic product. Packaging reduces the exposure of the hemostatic product to moisture as exposure to moisture can negatively impact the efficacy of the hemostatic mixture by causing the proteins to activate before the proteins are needed to achieve hemostasis.

To reduce the moisture concentration in the hemostatic mixture container, a desiccant may be placed in the hemostatic mixture container. A person of skill in the art will appreciate that a variety of desiccants may be selected based upon factors such as the initial moisture content of the hemostatic mixture.

The hemostatic mixture needs to be sterilized. Depending on the process that is used to fabricate the hemostatic product, sterilization can be done after the hemostatic mixture is packaged or after the hemostatic product is formed by applying the hemostatic mixture to the support material.

A variety of techniques may be used to sterilize the hemostatic mixture. The selected sterilization technique should minimize denaturation of the proteins. Even while the denaturation of the proteins is minimized, it is anticipated that at least some of the proteins will be denatured during the sterilization process. The denaturation should be less than about 20 percent.

The moisture level in the hemostatic mixture container can further be reduced by injecting an inert gas into the container. In certain embodiments, the inert gas is nitrogen. The processing reduces the moisture level in the hemostatic mixture container to less that about 6 percent, which is a significant reduction from the initial moisture level of the hemostatic mixture, which is about 11 percent depending on ambient conditions. In other embodiments, the moisture level in the hemostatic mixture container is less than about 3 percent. Another benefit of the nitrogen injection is that the nitrogen injection displaces oxygen from the container.

Another technique to minimize denaturation of the proteins in the hemostatic mixture is to maintain the hemostatic mixture at a low temperature prior to use. The temperature may be less than about 32° F. In other embodiments, the temperature is less than about 0° F. One technique that may be used to maintain the hemostatic mixture and/or the hemostatic product at the low temperature is placing dry ice adjacent to the hemostatic mixture and/or the hemostatic product.

The quantity of fibrinogen added to the hemostatic product may be adjusted by changing either the concentration of the fibrinogen in the hemostatic mixture or changing the rate at which the hemostatic mixture is used in the hemostatic product. The quantity of fibrinogen added to the hemostatic product is generally in the range of from about 10 milligrams to about 3 grams. In certain embodiments, the amount of fibrinogen in each of the hemostatic products is between about 20 milligrams to about 1 gram.

The quantity of thrombin added to the hemostatic product may be adjusted by changing either the concentration of the thrombin in the hemostatic mixture or changing the rate at which the hemostatic mixture is used in the hemostatic product. The quantity of thrombin added to each of the hemostatic products is generally between about 10 and 10,000 NIH Units. In certain embodiments, the amount of thrombin in each of the hemostatic products is between about 20 and 6,000 NIH Units.

In some embodiments, the therapeutic agents may themselves be electrospun. For example, the therapeutic agents are dissolved in and spun from a solution. In some embodiments, the therapeutic agents may be electrospun into fibers. In other embodiments, the active agents may be electrospun into other forms such as droplets, beads, etc.

In some applications, active agents such as thrombin may be electrosprayed with sucrose to form sugar droplets, which tends to stabilize thrombin and can also “trap” other substances of interest for delivery to the hemostatic product.

For thrombin and fibrinogen, in most embodiments, these (or other) active agents are in a finely dispersed dry, particulate or granular form e.g. as a fine powder or dust, as electrospinning may tend to decrease their activity. In other words, the active agents are not electrospun either by themselves.

Usually the agents are bioactive agents that have a beneficial or therapeutic effect at the wound site. In one embodiment, the site is a bleeding wound at which it is desired to form a blood clot to stop or slow the bleeding. In this embodiment, the therapeutic substances of interest may include, for example, thrombin and fibrinogen, although other agents active in promoting hemostasis, including but not limited to capscian, may also be included.

In addition, electrospun or non-electrospun collagen, agents that absorb water, various dry salts that would tend to absorb fluids when placed in contact with e.g. blood; engineered thrombin or thrombin mimics; engineered fibrinogen; agents that cause vasospasm (e.g. ADP, 5-hydroxytryptamine, 5-HT and thromboxane, (TXA-2) to help contract and seal a bleeding vessel, etc. may also be included.

Other components of the clotting cascade may be added to the hemostatic product, for example: tissue factors that are normally only expressed on the surface of damaged cells and that start the normal clotting cascade; serotonin which enhances platelet clumping and promotes vessel constriction; and other agents that are used to replace missing components of the clotting cascade in hemophilia, for example, factor 7 (which activates the so called external extrinsic coagulation cascade) and crude extracts of platelets.

Active agents that function to promote late stages of wound healing may also be included to, for example, facilitate cell migration and remodeling. The incorporation of collagen is an example of such an active agent.

One or more of any of these active agents may be used in the practice of the present invention. The therapeutic agents must be amenable to drying and are associated with the other components of the hemostatic product in the dry state, since liquid may negatively affect at least one of the components used in the hemostatic product. For example, the active agents may be desiccated or lyophilized, or water may be removed by other means.

Association of substances of interest with the electrospun dextran base may be accomplished by any of many suitable techniques that are known to those of skill in the art, and will depend in part on the precise form of the substance and the means at hand. For example, for powdered, particulate thrombin and fibrinogen, association may be carried out by sprinkling, shaking, blowing, etc. the agents onto a layer of the excipient or carrier.

In certain embodiments, the electrospun dextran base is placed on a vacuum table, which not only retains the electrospun dextran base in a substantially stationary position during the fabrication process but also causes the hemostatic agents to be drawn into the electrospun dextran base. This process thereby reduces the potential of the hemostatic agent becoming disassociated from the electrospun dextran base while stored in a package as well as when removed from the package prior to applying to the wound.

Depending on the density of the fiber mat, the substances of interest may become relatively evenly dispersed throughout the woven mat of fibers or may be largely confined to the topmost section of the fiber mat. If no backing is present, the latter embodiment is preferable to prevent the particulate substance of interest from falling through and out of the mat.

The association of substances of interest with the electrospun dextran base may be carried out according to many different arrangements. For example, a first layer of electrospun dextran may be formed, and one or more of the substances may be associated with the first layer. Then another second layer of electrospun dextran may be formed on top of the substance(s) of interest, and the same or other substances of interest may be associated with the second layer, and so on.

A final or outermost layer of electrospun dextran may be added to prevent the dislodgement of substances of interest from the layer(s) below. The number of layers of excipient that are used in the hemostatic product of the invention may vary widely, from as few as 1-2 to as many as several dozen, or even several hundred, depending on the desired characteristics of the hemostatic product.

Typically, a hemostatic product will contain 1-2 layers. In other embodiments the hemostatic product may include between 2-20 layers. The very slight amount of moisture that is present in a prepared hemostatic product may help to trap and retain the thrombin and fibrinogen on the surface of the hemostatic product.

The height or thickness of the hemostatic product can vary considerably depending on the intended use of the hemostatic product. In certain embodiments, the hemostatic product has a thickness of between about 1 millimeter and about 5 centimeters.

The thickness of the hemostatic product (which is related to the volume) may impact the rate of dissolution of the dextran upon contact with liquid. For example, a thin hemostatic product (e.g. about 2 millimeters) will dissolve more rapidly than a hemostatic product that is thicker, providing the loft of the fibers is comparable.

In most embodiments, dissolution of the dextran fibers is extremely rapid, e.g. about 5 minutes or less after exposure to liquid, or about 4 minutes or less, or about 3 minutes or less, or about 2 minutes or less, or about 1 minute or less. In certain embodiments, the hemostatic product substantially dissolves in between about 1 second and about 20 seconds.

This rapid dissolution may be referred to herein as “instantaneous” or “immediate” dissolution. Compression of an electrospun dextran mat may be used to modulate the rate of dissolution, with greater levels of compression inversely impacting the rate, i.e. generally, the greater the degree of compression, the slower the rate of dissolution.

The rapid rate of dissolution is advantageous, particularly when delivering biologically active agents (e.g. hemostatic agents) to a site of action such as a wound. Rapid dissolution of the carrier dextran fibers provides extremely rapid delivery of the hemostatic agents to the wound upon deployment of the hemostatic product.

Generally, the amount of water that is present in the substances when they are associated with the electrospun dextran fibers is less than about 5%, and preferably less than about 2%. These substances retain full or partial activity when rehydrated, e.g. in blood. Generally, therapeutic substances associated with the hemostatic products of the invention retain, upon contact with liquid, at least about 25%, or about 50%, or even about 75% to 100% of their activity before drying or desiccation, as compared to standard preparations of the substance using standard assays that are known to those of skill in the art.

If thrombin is included in the hemostatic product, it may be desirable to reduce the moisture content of the hemostatic product (e.g. a bandage or gauze) to less than about 5% to preserve thrombin activity during sterilization.

This moisture content reduction can be achieved by drying the fabricated hemostatic product, e.g., under a vacuum, or by using a fabrication method that reduces moisture content from the beginning.

The hemostatic product may include one or more stabilizers such as is described in U.S. application Ser. No. 13/622,690, which is assigned to the assignee of the present application and the contents of which are incorporated herein by reference. The stabilizers may enhance the ability of the hemostatic product to dissolve when the hemostatic products are applied to the injury site.

Prior to use of the hemostatic product, it may be desirable for the hemostatic product to be carried by a person on whom the hemostatic product could potentially be used and/or by a person who could potentially use the hemostatic product. In other embodiments, the hemostatic product resists degradation at temperatures of more than 140° F. to less than 0° F.

In certain embodiments, the hemostatic product should resist degradation when exposed to the elevated temperature such as up to about 150° F. for more than about 3 hours. In other embodiments, the hemostatic product should resist degradation when exposed to the elevated temperature for up to about 24 hours.

A threshold for the hemostatic product to be viewed as not experiencing degradation is that the hemostatic product does not exhibit noticeable visible physical changes when viewing the hemostatic product without magnification. The hemostatic product should also not experience noticeable physical changes when the hemostatic product is examined with magnification such as with a magnifying glass or a microscope.

The preceding characteristics should be displayed by the hemostatic product regardless of whether the hemostatic product is retained in the packaging materials while exposed to the elevated temperature conditions.

The stabilizer also enhances the usable shelf life of the hemostatic product. In certain embodiments, the stabilizer provides the hemostatic product with a shelf life of at least about 2 years. In other embodiments, the hemostatic product exhibits a shelf life of at least 3 years. As used herein, the term usable shelf life means that the hemostatic product does not exhibit noticeable degradation when viewed without magnification or with magnification such as a magnifying glass or microscope.

To minimize the potential of degradation of the hemostatic product, the hemostatic product should be protected from exposure to moisture because when the components used in the hemostatic product are exposed to moisture, the components degrade such as by dissolving.

In some embodiments of the invention, the hemostatic products also include one or more support structures or support materials incorporated therein. For example, a backing may be incorporated into the hemostatic product.

The support material may be formed from various electrospun materials such as polyglycolic acid (PGA), polylactic acid (PLA), and their copolymers (PLGAs); charged nylon, etc. In one embodiment, the support material is compressed electrospun dextran fibers. By “compressed electrospun dextran fibers,” it is meant that electrospun dextran fibers are compressed together under pressure.

The support material may or may not be soluble in liquid, or may be slowly soluble in liquid, and may or may not be permeable to liquid. Slowly soluble materials include those from which absorbable or dissolving (biodegradable) stitches or sutures are formed, included PGA, polylactic and caprolactone polymers.

In certain embodiments, the support material may dissolve relatively quickly such as less than about 1 hour. In other embodiments, the support material may dissolve within from about 10 days to 8 weeks. In either case, the support material provides the advantage of not having to remove the hemostatic product and risk disrupting the clot.

However, in any case, the support material should not interfere with the immediate dissolution of the hemostatic product and delivery of the active agents associated therewith into the liquid that dissolves the hemostatic product. All such arrangements, shapes, and embodiments of carrier layers and support materials as described herein are intended to be encompassed by the invention.

The hemostatic product may be sterilized prior to use, generally by using electromagnetic radiation, for example, X-rays, gamma rays, ultraviolet light, etc. Typically, the hemostatic products are sterilized using X-rays in a dose of about 5 kilograys (kGray). Any method that does not destroy the carrier or the activity of substances associated with the fibers may be used to sterilize the hemostatic products of the invention.

The hemostatic product may also include diagnostic agents that can be used by the treating medical professional to diagnose the nature of the injury. In certain embodiments, the diagnostic agent may change colors to indicate the presence of particular chemicals in the blood or to indicate particular characteristics of the blood. For example, if the patient is currently taking medications that cause thinning of the patient's blood. The diagnostic agents could also change colors to indicate the oxygen and/or glucose level of the blood.

In other embodiments, the products of the invention need not comprise agents that promote clotting at all. Those of skill in the art will recognize that the products of the invention are highly suitable for delivering many substances of interest to a desired liquid environment or location. For example, the products may be designed for delivery of therapeutic or beneficial substances to any moist environment of the body, where there is sufficient liquid to dissolve the electrospun dextran fibers and release the active substance, and where dissolved dextran is not problematic.

Such substances may include, for example, enzymes or their precursors (e.g. pro-enzymes or zymogens) and their substrates, substances that activate a protein or enzyme (e.g. proteases, cofactors, etc.), and the like.

For example, hemostatic products comprised of only thrombin might be used for small injuries or in combination with other interventions. In addition, other therapeutically beneficial substances may also be associated with the hemostatic product, including but not limited to: antibiotics, antiviral agents, anti-helminthic agents, anti-fungal agents, medicaments that alleviate pain, growth factors, bone morphogenic protein, vasoactive materials (e.g. substances that cause vasospasms), steroids to reduce inflammation, chemotherapy agents, contraceptives, etc.

Examples include but are not limited to oral, nasal, tracheal, anal, lung, and vaginal delivery of substances such as anti-microbial agents, analgesic agents, nutritional agents, etc. Oral applications include the delivery of substances useful for dental treatments, e.g. antibiotics, pain medications, whitening agents, etc.

In some embodiments, no bodily fluid is present (or if insufficient body fluid is present) and the applied hemostatic product can be “activated” by wetting, e.g. by spraying, or by otherwise applying a source of moisture (e.g. by exposing the hemostatic product to a moist material such as a sponge), or dropping hemostatic products into a liquid (e.g. a body of water), to cause release of the agents of interest associated with the dextran fibers.

In addition to being used to produce hemostasis in humans, the concepts of the invention may be adapted for use in conjunction with other animals. Examples of such animals on which the invention can be used include dogs and cats.

In the preceding detailed description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The preceding detailed description, therefore, is not to be taken in a limiting sense, and the scope of the present invention is defined by the appended claims.

It is contemplated that features disclosed in this application, as well as those described in the above applications incorporated by reference, can be mixed and matched to suit particular circumstances. Various other modifications and changes will be apparent to those of ordinary skill.

1. A method of forming a hemostatic product comprising:
preparing a porous base that is capable of substantially dissolving when applied to a bleeding wound;
mixing a hemostatic agent with at least one salt to prepare a hemostatic mixture; and
dispersing the hemostatic mixture on the porous base to form the hemostatic product.

2. The method of claim 1, wherein the porous base comprises electrospun dextran and wherein the hemostatic agent comprises at least one of thrombin and fibrinogen.

3. The method of claim 1, wherein the method further comprises:
forming a thrombin mixture that comprises thrombin and at least one salt;
forming a fibrinogen mixture that comprises fibrinogen and at least one salt; and
mixing the thrombin mixture and the fibrinogen mixture to form the hemostatic mixture.

4. The method of claim 3, and further comprising reducing a particle size of at least one of the thrombin mixture and the fibrinogen mixture to less than about 0.75 millimeters.

5. The method of claim 1, wherein the at least one salt comprises sodium chloride, trisodium citrate or combination thereof.

6. The method of claim 1, wherein the at least one salt is provided at a concentration of between about 10 percent by weight and about 70 percent by weight of the hemostatic mixture.

7. The method of claim 1, and further comprising mixing at least one buffer into the hemostatic mixture.

8. The method of claim 7, wherein the at least one buffer is bis(2-hydroxyethyl)amino-tris(hydroxymethyl)methane.

9. The method of claim 7, wherein the at least one buffer is provided at a concentration of between about 15 percent by weight and about 35 percent by weight of the hemostatic mixture.

10. The method of claim 1, and further comprising:
packaging the hemostatic mixture in a moisture controlled container before the hemostatic mixture is dispersed on the porous base; and
purging an interior of a package in which the hemostatic mixture is placed with an inert gas.

11. The method of claim 1, and further comprising maintaining the packaged hemostatic mixture at a temperature of less than about 32° F. prior to dispersing the hemostatic mixture on the porous base.

12. The method of claim 1, and further comprising mixing at least one biologically tolerable polymer into the hemostatic mixture.

13. The method of claim 12, wherein the at least one biologically tolerable polymer is poly(ethylene glycol).

14. The method of claim 12, wherein the at least one biologically tolerable polymer is provided at a concentration of between about 13 percent by weight and about 20 percent by weight of the hemostatic mixture.

15. A method of forming a hemostatic product comprising:
preparing a porous base that is capable of substantially dissolving when applied to a bleeding wound;
mixing a hemostatic agent with at least one biologically tolerable polymer to prepare a hemostatic mixture; and
dispersing the hemostatic mixture on the porous base to form the hemostatic product.

16. The method of claim 15, wherein the at least one biologically tolerable polymer is poly(ethylene glycol).

17. The method of claim 15, wherein the at least one biologically tolerable polymer is provided at a concentration of between about 13 percent by weight and about 20 percent by weight of the hemostatic mixture.

18. The method of claim 15, and further comprising mixing at least one buffer into the hemostatic mixture.

19. The method of claim 18, wherein the at least one buffer is bis(2-hydroxyethyl)amino-tris(hydroxymethyl)methane and wherein the at least one buffer is provided at a concentration of between about 15 percent by weight and about 35 percent by weight of the hemostatic mixture.

20. The method of claim 15, wherein the porous base comprises electrospun dextran and wherein the hemostatic agent comprises at least one of thrombin and fibrinogen.

21. A method of forming a hemostatic product comprising:
preparing a porous base that is capable of substantially dissolving when applied to a bleeding wound;
providing a thrombin component;
providing a fibrinogen component;
reducing a particle size of at least one of the thrombin component and the fibrinogen component to less than about 0.75 millimeters;
mixture the thrombin component and the fibrinogen component to form a hemostatic mixture; and
dispersing the hemostatic mixture on the porous base to form the hemostatic product.

22. The method of claim 21, wherein reducing the particle size is done by sifting.
